(19) United States
(12) Patent Application Publication
(10) Pub. No.: US 2006/0070145 A1
(43) Pub. Date: Mar. 30, 2006

(54) Manufactured seed having a live end seal

(76) Inventors: William C. Carlson, Olympia, WA (US); Jessie L. Wetzbarger, Tacoma, WA (US); Jeffrey E. Hartle, Tacoma, WA (US)

Correspondence Address: WEYERHAEUSER COMPANY;INTELLECTUAL PROPERTY DEPT., CH 1J27, P.O. BOX 9777, FEDERAL WAY, WA, 98063, US

(21) Appl. No.: 11/217,885

(22) Filed: Sep. 1, 2005

Related U.S. Application Data
(60) Provisional application No. 60/613,598, filed on Sep. 27, 2004.

(51) Int. Cl. A01H 5/10

(52) U.S. Cl. 800/298

(57) ABSTRACT

An artificial seed (20) is provided. The artificial seed includes a seed shell (24) sized and configured to receive an embryo (42) therein. The artificial seed also includes a seal disposed on a surface of the seed shell, and the seal includes a least an inner sealing layer (44) and an outer sealing layer (60). The outer sealing layer is hydrophilic.

### CROSS REFERENCE TO RELATED APPLICATION

The present application claims the benefit of U.S. Provisional Application No. 60/613,598, filed Sep. 27, 2004.

### FIELD OF THE INVENTION

The present invention relates generally to artificial seeds and, more particularly, to live end seals for manufactured seeds.

### BACKGROUND OF THE INVENTION

Asexual propagation of plants has been shown for some species to yield large numbers of genetically identical embryos, each having a capacity to develop into a normal plant. Such embryos are usually further cultured under laboratory conditions until they reach an autotrophic “seedling” state characterized by an ability to produce its own food via photosynthesis, resist desiccation, produce roots able to penetrate soil, and fend off soil microorganisms. Some researchers have experimented with the production of artificial seeds, known as manufactured seeds, in which individual plant somatic or zygotic embryos are encapsulated in a seed coat. Examples of such manufactured seeds are disclosed in U.S. Pat. No. 5,701,699, issued to Carlson et al., the disclosure of which is hereby expressly incorporated by reference.

Typical manufactured seeds include a seed shell, synthetic gametophyte and a plant embryo. A manufactured seed that does not include the plant embryo is known in the art as a “seed blank.” The seed blank typically is a cylindrical capsule having a closed end and an open end. The synthetic gametophyte is placed within the seed shell to substantially fill the interior of the seed shell. A longitudinally extending hard porous insert, known as a cotyledon restraint, may be centrally located within one end of the seed shell, surrounded by the synthetic gametophyte, and includes a centrally located cavity extending partially through the length of the cotyledon restraint.

The cavity is sized to receive the plant embryo therein. The well-known plant embryo includes a radicle end and a cotyledon end. The plant embryo is deposited within the cavity of the cotyledon restraint, cotyledon end first. The plant embryo is then sealed within the seed blank by an end seal. There is a weakened spot in the end seal to allow the radicle end of the plant embryo to penetrate the end seal.

In the past, the end seal is attached to the manufactured seed by either stretching a wax base film, such as Parafilm®, or forming a wax seal to enclose the embryo within the manufactured seed. Although such types of end seals are successful in sealing the embryo within the manufactured seed, they are not without their problems. As a non-limiting example, such end seals work well in laboratory conditions but can prematurely break when placed in more rigorous handling environments, such as agricultural sowers. Additionally, to protect against microbial invasion, such end seals have been treated with a tribiotic ointment. Such a treatment further reduces the strength of the end seal. Thus, there exists a need for a tertiary end seal for manufactured seeds that protects the secondary end seal.

### SUMMARY OF THE INVENTION

An artificial seed is provided. The artificial seed includes a seed shell and an embryo disposed within the seed shell. The artificial seed also includes a seal disposed on a surface of the seed shell, wherein the seal includes at least an inner sealing layer and an outer sealing layer of a hydrophilic material. In one embodiment of the present invention, the outer sealing layer is a cellulose-based material. In certain embodiments, the seal is an end seal disposed on one end of the seed shell. In yet another embodiment of the present invention, the seal is disposed on the sidewalls of the seed shell.

An artificial seed formed in accordance with the various embodiments of the present invention have several advantages over currently available manufactured seeds. In that regard, the outer sealing layer functions by protecting the inner sealing layer during seed handling and sowing. Because the outer sealing layer has a high affinity for water and swells when hydrated, it softens during irrigation following sowing to allow the outer sealing layer to break, thereby facilitating germination through both the inner and outer sealing layers. Additionally, the outer sealing layer is suitable as a carrier for pesticides that further protect the embryo prior to and during germination. As germination occurs through the softened outer sealing layer, the pesticides remain functional as the outer sealing layer is penetrated.

### BRIEF DESCRIPTION OF THE DRAWINGS

The foregoing aspects and many of the attendant advantages of this invention will become better understood by reference to the following detailed description, when taken in conjunction with the accompanying drawings, wherein:

FIG. 1 is a cross-sectional side planar view of an artificial seed formed in accordance with one embodiment of the present invention, showing the artificial seed having a primary, secondary and tertiary end seal;

FIG. 2 is a partial, cross-sectional side planar view of the artificial seed of FIG. 1 showing application of an antimicrobial agent to a germinating embryo as it penetrates the secondary and tertiary end seals; and

FIG. 3 is a side planar view of an alternate embodiment of the manufactured seed of FIG. 1, showing the tertiary end seal applied to both the secondary end seal and sidewalls of the manufactured seed.

### DETAILED DESCRIPTION OF THE PREFERRED EMBODIMENT

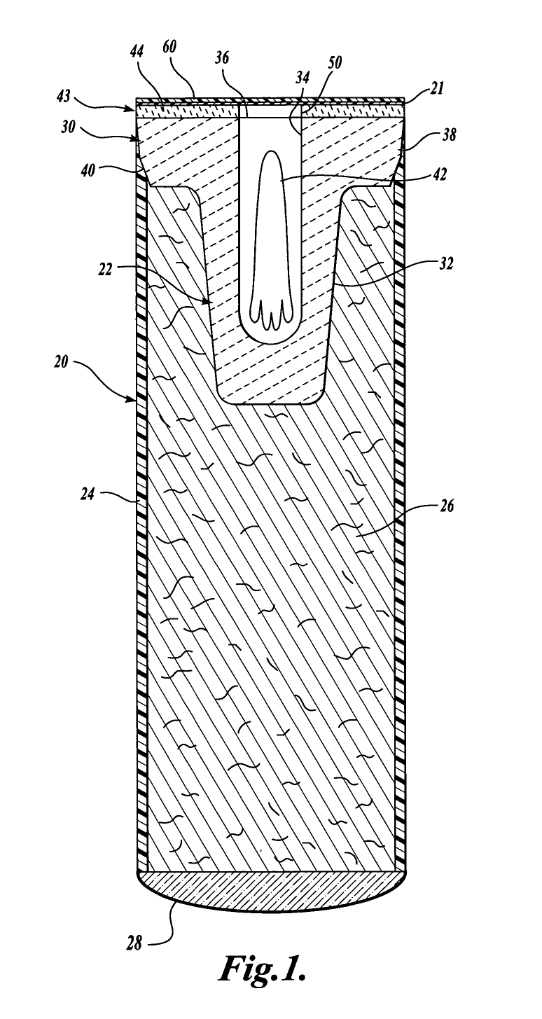

FIG. 1 illustrates an artificial seed 20 having a tertiary end seal 60 constructed in accordance with one embodiment of the present invention. The artificial seed 20 includes a cylcap 22, a seed shell 24, a nutritive media 26, such as a gametophyte, and a dead end seal 28. The seed shell 24 is suitably formed from a section of tubular material. In one embodiment, the seed shell 24 is a sectioned straw of fibrous material, such as paper. The sections of straw may be pre-treated in a suitable coating material, such as wax.

In other embodiments, the seed shell 24 is formed from a tubular section of biodegradable, plastic material. One such material is a utilized polylatic acid (“PLA”) and is sold by NAT-UR of Los Angeles, Calif. Such biodegradable plastic tubes are similarly sectioned into appropriate lengths for a manufactured seed. Further, such biodegradable plastic tubes do not require a wax coating as such tubes are already resistive to environmental elements. It should be apparent that although sectioning tubes is preferred, other embodiments, such as obtaining tubes of appropriate size for use as manufactured seeds, are also within the scope of the present invention.

The cylcap 22, also known as a restraint, is suitably manufactured from a porous material having a hardness strong enough to resist puncture or fracture by a germinating embryo, such as a ceramic or porcelain material, and includes an end seal portion 30 and a cotyledon restraint portion 32. The cotyledon restraint portion 32 is suitably integrally or unitarily formed with the end seal portion 30. The cylcap 22 also includes a longitudinally extending cavity 34 extending through the end seal portion 30 and partially through one end of cotyledon restraint portion 32. The open end of the cavity 34 is known as a cotyledon restraint opening 36. The cavity 34 is sized to receive a plant embryo 42 therein.

In certain embodiments, as the cylcap 22 is suitably manufactured from a porous material, it may be desirable to coat the cylcap 22 with a barrier material to reduce the rate of water loss and restrict or reduce microbial entry. Such barriers include wax, polyurethane, glaze, nail polish, and a coating sold by Airproducts Airflex 4514.

The end seal portion 30 is suitably circular when viewed in a top planar view and includes sidewalls 38. Although circular is the preferred embodiment of the end seal portion 30, other embodiments and shapes, such as polygonal, square, triangular, oval and other shapes, are also within the scope of the present invention.

In the embodiment of FIG. 1, the sidewalls 38 are defined by the thickness of the end seal portion 30 and has a diameter substantially equal to the inside diameter of the seed shell 24. In certain embodiments, the cylcap 22 is bonded to the seed shell 24 by heat. As a non-limiting example, during manufacturing, the cylcap 22 may be heated to a predetermined temperature, such that when the seed shell 24 and the cylcap 22 are co-joined, heat transferred between the cylcap 22 and the seed shell 24 causes either the seed shell 24, the cylcap 22, or both to melt, thereby bonding the two together. Other methods of bonding the cylcap 22 to the seed shell 24, such as a wax bond or a hot glue melt, are also within the scope of the present invention.

The sidewalls 38 may include a tapered portion 40. The tapered portion 40 may be a chamfer of one end of the end seal portion 30. The tapered portion 40 assists in assembling the cylcap 22 to the seed coat 24 during manufacturing. Although a tapered portion 40 is preferred, other embodiments, such as a cylcap that does not include a tapered portion, are also within the scope of the present invention. An embryo 42 is disposed within the cavity 34 and is suitably sealed therein by a live end seal 43.

The live end seal 43 includes a primary end seal 44 and a secondary end seal 21. The primary end seal 44 is suitably formed from a PLA material described above and includes a centrally located opening 50. The opening 50 is sized to correspond to diameter of the cavity 34 of the cylcap 22 to permit a germinating embryo 42 to pass therethrough. The primary end seal 44 is suitably attached to the end seal portion 30 by a variety of methods, including glue or heat bonding.

As a non-limiting example, the primary end seal 44 is mated to a pre-heated cylcap 22, such that the opening 50 is located above the cavity 34. The heat welds or bonds the primary end seal 44 to the cylcap 22. It should be apparent that the primary end seal 44 may be attached to the cylcap 22 before or after the cylcap 22 is attached to the seed shell 24. Also, if the seed shell 24 is constructed from PLA, it is desirable but not necessary that the melt temperature of the primary end seal 44 and the seed shell 24 be similar.

As another non-limiting example of attaching the primary end seal 44 to the cylcap 22, includes an adhesive gasket. In this example, the primary end seal 44 is heat sealed or bonded to the cylcap 22 with the opening 50 co-axially aligned with the cavity 34. In this process, a form is used to bend edges of the primary end seal 44 around the perimeter of the end seal portion 30 of the cylcap 22. If the melt temperature of the primary end seal 44 and the seed shell 24 are different, then a low bloom cyanoacrylate is used as an adhesive gasket to bond the primary end seal 44 and the seed shell 22.

Heat is applied after the glue and is done so as to thin the glue seal by melting incongruities that typically occur when manufacturing the seed shell 24 and forming the adhesive joint. Thereafter, the cylcap 22, including the primary end seal 44, is attached to the seed shell 24. As noted above, this method is also suitable to a cylcap 22 that is already attached to the seed shell 24. Finally, the foregoing method of attaching a primary end seal 44 to a seed shell 24 may be used for heat weld compatible or incompatible materials.

The secondary end seal 21 will now be described in greater detail. In that regard, the secondary end seal 21 is suitably formed from a well-known sealing material, such as Parafilm®. The secondary end seal 21 is formed and attached to the primary end seal 44 by a well-known method, such as heat bonding or gluing. The secondary end seal 21 also includes a predetermined burst strength to permit a germinating embryo 42 to penetrate through the live end seal 44.

Still referring to FIG. 1, the tertiary end seal 60 will now be described in greater detail. The tertiary end seal 60 and live end seal 43, as used in the present embodiment, define an outer sealing layer and an inner sealing layer, respectively. Although the live end seal 43 has been described as including both a primary end seal 44 and a second end seal 21, it should be apparent that the invention is not intended to be so limited. As a non-limiting example, the live end seal 43 may include only the secondary end seal 21 and, therefore, such embodiments are also within the scope of the present invention.

The combination of the tertiary end seal 60 and live end seal 43 creates a sealing surface, wherein the sealing layer, defined by the tertiary end seal 60, is made from a predetermined material that degrades in structural integrity after a predetermined exposure to environmental conditions. The tertiary end seal 60 also serves as an anti-microbial sealant to seal and protect around the embryo as the embryo germinates and emerges from within the seed shell 24 and protects the cotyledon restraint cavity.

The tertiary end seal 60 is suitably manufactured from a hydroxypropylmethylcellulose. Other types of hydrophilic materials and cellulose-based coatings include cellulose acetate phthalate, hydroxypropylethylcellulose, ethylcellulose, methylcellulose, microcrystalline cellulose, and carrageenan. Such materials have the desired properties of having a relatively high structural integrity when dry and such structural integrity degrades when exposed to environmental conditions, such as water.

In certain embodiments, it is desirable to add an anti-microbial agent, such as Thiram 50WP. Any anti-microbial agent that is substantially non-phytotoxic at the desired concentration is also within the scope of the present invention. As is described in greater detail below, a tertiary end seal 60 treated with an anti-microbial agent is suitable as a carrier for pesticides to protect the embryo 42 prior to and during germination.

The break-through strength of the tertiary end seal 60 is a function of the polymer used and the amount of it used to create the tertiary end seal 60. As a non-limiting example, breaking strength was tested using a tertiary end seal 60 manufactured from hydroxypropylmethylcellulose (HPMC) treated with Thiram 50WP as the anti-microbial agent. A test was conducted to determine the breaking strength of various mixtures. In that regard, a total of six treatments, as set forth below, were tested for break-through strength. A mixture of 2.64 g of HPMC 120 and 0.36 g HPMC 4000 was created for use in treatments 1 and 2.

Treatment 1 used a 0.91 g HPMC mix plus 0.4823 g Thiram and 8.61 ml of water, resulting in a 9.1% HPMC mix by weight.

Treatment 2 used 1.25 g HPMC mix plus 0.4823 g Thiram and 8.27 ml of water, resulting in 12.5% HPMC mix by weight.

Treatment 3 included 0.91 g HPMC 4000 plus 0.4823 g Thiram and 8.61 ml of water, resulting in 9.1% HPMC 4000 by weight.

Treatment 4 utilized 0.86 g HPMC 4000 plus 0.4823 g Thiram and 8.66 ml of water.

Treatment 5 utilized a mechanically disturbed lid attached to the seed.

Treatment 6 used a mechanically disturbed lid attached to the seed and then coated with a tribiotic ointment and left for 24 hours before testing. In this case, the secondary end seal has been slightly disturbed with an abrasion pad scrubber to allow the tertiary end seal to be glued to the primary end seal.

Treatments 1-4 were done on top of the seed made as in treatment 6.

Twelve seeds per treatment were tested after coating and drying, and another twelve were tested 1 to 1.5 hours after they were rewetted with water. Table 1, set forth below, sets forth the results.

TABLE 1

| Treatment | 1 Dry | 1 Wet | 2 Dry | 2 Wet | 3 Dry | 3 Wet | 4 Dry | 4 Wet | 5 | 6 |
|---|---|---|---|---|---|---|---|---|---|---|
| Mean Breaking | 45.50 | 1.23 | 454.10 | 1.84 | 575.16 | 1.87 | 567.08 | 2.07 | 16.06 | 1.3 |
| Strength (g) | | | | | | | | | | |
| Standard Error | 5.06 | 0.14 | 45.3 | 0.60 | 8.34 | 0.27 | 15.7 | 0.73 | 2.23 | 0.15 |

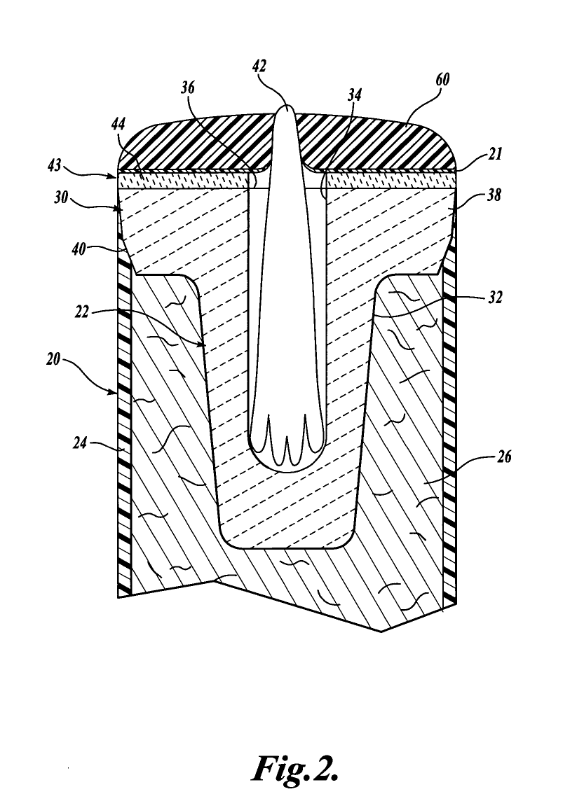

As may be best seen by referring to FIG. 2, as the embryo 42 germinates, it perforates both the live end seal 43 and tertiary end seal 60. Because the tertiary end seal 60 includes an anti-microbial agent, as the embryo 42 penetrates through the tertiary end seal 60, a residue of the anti-microbial agent coats at least the sides of the embryo 42 during germination.

When the artificial seed 20 is handled and sowed, the tertiary end seal 60 protects the live end seal 43 from damage associated with such activities. The tertiary end seal 60 softens during irrigation following sowing to allow the live end seal 43 to break at the desired level during germination. The tertiary end seal 60 softens when exposed to water due to the hydrophilic properties of the materials used to manufacture the tertiary end seal 60. As a result, the structural integrity of the tertiary end seal 60 degrades when exposed to various environmental conditions, while initially maintaining its structural integrity during handling and sowing.

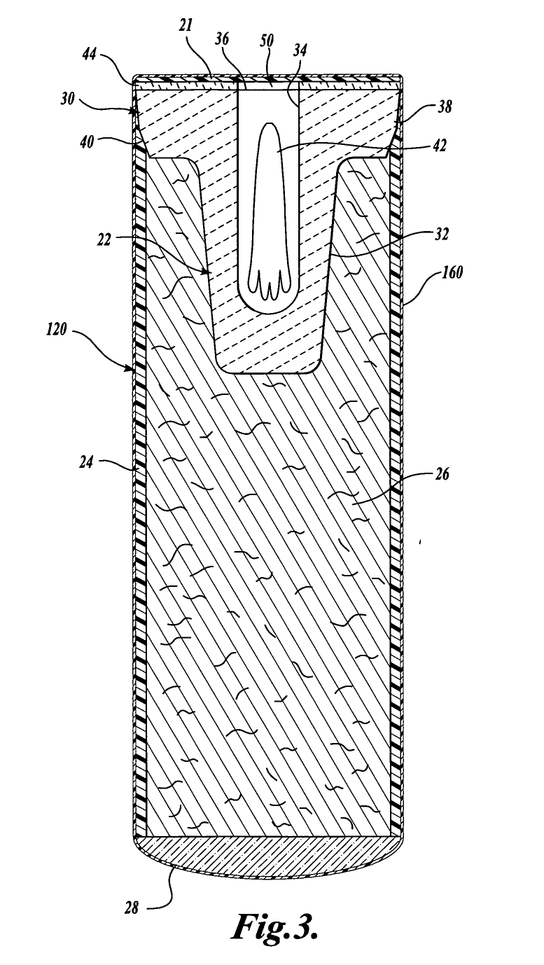

Referring to FIG. 3, an alternate embodiment of the artificial seed of FIGS. 1 and 2 will now be described in greater detail. The artificial seed 120 of FIG. 3 is substantially identical in materials and operation as the first embodiment described above, with the exception that the same material used to form the tertiary end seal 160 is applied to the entire perimeter of the artificial seed. In that regard, after an artificial seed is assembled, a layer of hydrophilic material described above for the first embodiment may be applied to the entire outside surface of the artificial seed 120. Further, the hydrophilic material may include an anti-microbial agent, such as those described above.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. An artificial seed, comprising:
(a) a seed shell;
(b) a restraint disposed within the seed shell, the restraint having a cavity;
(c) an embryo disposed within the cavity; and
(d) a seal disposed on a surface of the seed shell, the seal including at least an inner sealing layer and an outer sealing layer, wherein outer sealing layer is hydrophilic.

2. The artificial seed of claim 1, wherein the outer sealing layer includes an anti-microbial agent.

3. The artificial seed of claim 2, wherein the anti-microbial agent is applied to the embryo as the embryo penetrates the seal.

4. The artificial seed of claim 1, wherein the outer sealing layer maintains an anti-microbial seal as the embryo germinates and emerges from within the seed shell.

5. The artificial seed of claim 1, wherein the outer sealing layer is a cellulose based material.

6. The artificial seed of claim 1, wherein the outer sealing layer is manufactured from a group of materials consisting of cellulose acetate phthalate, hydroxypropylethylcellulose, ethylcellulose, methylcellulose, microcrystalline cellulose, and carrageenan.

7. The artificial seed of claim 1, wherein the seal is an end seal disposed on one end of the seed shell and substantially seals the embryo within the cavity.

8. The artificial seed of claim 7, wherein the outer sealing layer is disposed on sidewalls of the seed shell.

9. An artificial seed, comprising:
(a) a seed shell having an embryo disposed therein; and
(b) a sealing surface attached to the seed shell, the sealing surface having at least a sealing layer of a predetermined material that degrades in structural integrity after a predetermined exposure to environmental conditions.

10. The artificial seed of claim 9, wherein the sealing surface includes an end seal attached to the seed shell and disposed beneath the sealing layer.

11. The artificial seed of claim 10, wherein at least the sealing layer includes an anti-microbial agent.

12. The artificial seed of claim 11, wherein the anti-microbial agent is applied to the embryo as the embryo penetrates the sealing layer.

13. The artificial seed of claim 9, wherein the sealing layer maintains an anti-microbial seal as the embryo germinates and emerges from within the seed shell.

14. The artificial seed of claim 9, further comprising a second sealing layer attached to sidewalls of the seed shell.

15. The artificial seed of claim 14, wherein the sealing layer and the second sealing layer are hydrophilic.
